SEQUENCE LISTING (10 sequences)

SEQ ID NO: 1 - DNA, 411 bp, synthetic construct
  source 1..411: mol_type = other DNA; note = sequence encoding the S1B protein
aatattactgttagtgctgcttttggtggtcatagtggtgctaatttaattgctagtgatactactattaatggttttagtagtttttgtgttgatactcggcaatttacgattactttattttataatgttactaatagttatggttatgttagtaaaagtcaagatagtaattgtccatttacgttacaaagtgttaatgattatttatcatttagtaaattttgtgttagtactagtttattagccggtgcttgtactattgatttatttggttatccagaatttggttcaggtgttaaatttactagtttatattttcaatttactaaaggtgaattaattactggtactccaaaaccattacaaggtgttactgatgttagttttatgcatcatcatcatcaccat

SEQ ID NO: 2 - DNA, 116 bp, synthetic construct
  source 1..116: mol_type = other DNA; note = PpepN promoter
ctgtcagtagacagtttttttaataagttaaagaaaagatgtaatttttctttgtactcgaaattttctattcaatttgatataattatattaatactgaatatttaggagaaggc

SEQ ID NO: 3 - DNA, 114 bp, synthetic construct
  source 1..114: mol_type = other DNA; note = sequence encoding the Usp45 secretion signal peptide
atgaaaaaaaagattatctcagctattttaatgtctacagtgatactttctgctgcagccccgttgtcaggtgtttacgctgacacaaactcagatattgctaaacaagatgcg

SEQ ID NO: 4 - DNA, 41 bp, synthetic construct
  source 1..41: mol_type = other DNA; note = primer PpepN-F
ataaggaggcactcaccatggctgtcagtagacagtttttt

SEQ ID NO: 5 - DNA, 52 bp, synthetic construct
  source 1..52: mol_type = other DNA; note = primer PpepN-R
gagataatcttttttttcatgccttctcctaaatattcagtattaatataat

SEQ ID NO: 6 - DNA, 40 bp, synthetic construct
  source 1..40: mol_type = other DNA; note = primer Usp45-F
atattcggaggaattttgaaatgaaaaaaaagattatctc

SEQ ID NO: 7 - DNA, 40 bp, synthetic construct
  source 1..40: mol_type = other DNA; note = primer Usp45-R
tcgtcgtcatccttgtaatccgcatcttgtttagcaatat

SEQ ID NO: 8 - DNA, 230 bp, synthetic construct
  source 1..230: mol_type = other DNA; note = sequence of the PpepN-Usp45 long fragment
ctgtcagtagacagtttttttaataagttaaagaaaagatgtaatttttctttgtactcgaaattttctattcaatttgatataattatattaatactgaatatttaggagaaggcatgaaaaaaaagattatctcagctattttaatgtctacagtgatactttctgctgcagccccgttgtcaggtgtttacgctgacacaaactcagatattgctaaacaagatgcg

SEQ ID NO: 9 - DNA, 49 bp, synthetic construct
  source 1..49: mol_type = other DNA; note = primer S1B-F
gattacaaggatgacgacgataagaatattactgttagtgctgcttttg

SEQ ID NO: 10 - DNA, 22 bp, synthetic construct
  source 1..22: mol_type = other DNA; note = primer S1B-R
ttaatggtgatgatgatgatgc

(19) United States
(12) Patent Application Publication
(10) Pub. No.: US 2025/0375516 A1
(43) Pub. Date: Dec. 11, 2025

(54) RECOMBINANT VECTOR FOR EXPRESSING IMMUNOGENIC PROTEIN OF PORCINE EPIDEMIC DIARRHEA VIRUS (PEDV), RECOMBINANT STRAIN INCLUDING THE SAME, AND USE THEREOF

(71) Applicant: CHANGSHA LVYE BIOTECHNOLOGY CO., LTD., Changsha (CN)

(72) Inventors: Yuanyuan ZHU, Changsha (CN); Wei DONG, Changsha (CN); Fan LIAO, Changsha (CN); Xiaohong XIE, Changsha (CN); Jiayi HE, Changsha (CN); Qing LI, Changsha (CN); Lixin WEN, Changsha (CN)

(73) Assignee: CHANGSHA LVYE BIOTECHNOLOGY CO., LTD., Changsha (CN)

(21) Appl. No.: 19/075,847

(22) Filed: Mar. 11, 2025

(30) Foreign Application Priority Data
Jun. 5, 2024 (CN) 202410719628.0

(51) Int. Cl. A61K 39/225; A61K 39/00; A61P 31/14; C07K 14/005; C12N 1/20; C12N 7/00; C12N 15/74; C12R 1/46

(52) U.S. Cl. CPC A61K 39/225; A61P 31/14; C07K 14/005; C12N 1/20; C12N 7/00; C12N 15/746; A61K 2039/523; A61K 2039/545; A61K 2039/552; C12N 2770/20022; C12N 2770/20034; C12R 2001/46

(57) ABSTRACT

A recombinant vector for expressing an immunogenic protein of porcine epidemic diarrhea virus (PEDV), a recombinant strain including the same and use thereof are provided. The recombinant vector for expressing the immunogenic protein S1B of the PEDV is provided, and the recombinant vector can be used to construct a recombinant Lactococcus lactis strain expressing the immunogenic protein S1B of the PEDV. The recombinant Lactococcus lactis strain and an expressed antigen protein S1B of the PEDV are mixed to prepare a microbial agent capable of preventing PEDV infection. The created microbial preparation may safely, effectively, and quickly prevent the PEDV infection in pigs and does not involve an immunization process.

### CROSS REFERENCE TO THE RELATED APPLICATIONS

This application is based upon and claims priority to Chinese Patent Application No. 202410719628.0, filed on Jun. 5, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named WGJB0209_Sequence_Listing.xml, created on Feb. 21, 2025, and is 12,521 bytes in size.

### TECHNICAL FIELD

The present disclosure belongs to the technical field of genetic engineering, and specifically relates to a recombinant vector for expressing an immunogenic protein of porcine epidemic diarrhea virus (PEDV), a recombinant strain including the same, and use thereof.

### BACKGROUND

Porcine epidemic diarrhea (PED) is a highly contagious disease caused by the porcine epidemic diarrhea virus (PEDV), with vomiting, diarrhea, and severe dehydration as the main clinical symptoms. Clinically, inactivated vaccines and attenuated virus vaccines for PEDV have been developed. Although these vaccines reduce mortality rates, the genetic diversity and complexity of prevalent PEDV strains, which include at least five subtypes with limited cross-protection, mean that the vaccines currently in use cannot effectively control viral infection and transmission, resulting in poor efficacy. Moreover, since the immune system of neonatal piglets is immature, traditional vaccination cannot provide timely protective effects, leading to immune failure. Although oral immunization is believed to induce intestinal mucosal immunity, gastrointestinal degradation mediated by gastric acid and proteases can lead to significant antigen loss, resulting in insufficient protection against PEDV infection.

At present, the safest, most economical, and most effective means of PEDV control involves biosecurity practices, with the principle of preventing contact between the virus and the host. However, existing methods cannot guarantee complete separation between the virus and the host.

### SUMMARY

An objective of the embodiments of the present disclosure is to provide a recombinant vector for expressing an immunogenic protein of PEDV, a recombinant strain including the same, and use thereof. In the present disclosure, a recombinant bacterial expression system for expressing a PED S1B protein is constructed, providing a theoretical basis for the development of a microbial preparation for blocking PEDV infection.

The present disclosure provides a recombinant vector for expressing an immunogenic protein of PEDV, including a plasmid vector and a nucleotide sequence encoding an S1B protein.

In some embodiments, the plasmid vector includes PNZ8149-Pnis-PpepN-Usp45; and

the PNZ8149-Pnis-PpepN-Usp45 is based on a PNZ8149 vector and includes a nucleotide sequence encoding a PpepN promoter and a nucleotide sequence encoding a Usp45 secretion signal peptide that are ligated in sequence; the nucleotide sequence encoding the PpepN promoter and the nucleotide sequence encoding the Usp45 secretion signal peptide that are ligated in sequence are located downstream of an original promoter of the PNZ8149 vector; the nucleotide sequence encoding the PpepN promoter is set forth in SEQ ID NO: 2; and the nucleotide sequence encoding the Usp45 secretion signal peptide is set forth in SEQ ID NO: 3.

In some embodiments, the nucleotide sequence encoding the S1B protein is located between restriction sites Nco I and Sac I of the PNZ8149-Pnis-PpepN-Usp45.

The present disclosure further provides a recombinant strain expressing an immunogenic protein of a PEDV, including the recombinant vector.

In some embodiments, a host strain for the recombinant strain includes Lactococcus lactis.

In some embodiments, the Lactococcus lactis includes Lactococcus lactis NZ3900.

The present disclosure further provides a microbial agent for preventing PEDV infection, including the recombinant strain as an active ingredient.

In some embodiments, the recombinant strain in the microbial agent has a viable count of 2.0-3.0×10⁹ colony forming unit (CFU)/mL to 2.0-3.0×10¹⁰ CFU/mL.

The present disclosure further provides use of the recombinant vector, the recombinant strain, or the microbial agent in preparation of a drug for preventing PEDV infection.

In some embodiments, the drug includes a drug for blocking binding of the PEDV to a cell receptor.

### Beneficial Effect

The present disclosure provides a recombinant vector for expressing the immunogenic protein S1B of PEDV, which may be used to construct a recombinant Lactococcus lactis strain expressing the immunogenic protein S1B of PEDV. The recombinant Lactococcus lactis strain expressing the antigen protein S1B of PEDV are mixed to prepare a microbial agent capable of preventing PEDV infection. In the present disclosure, after the pig takes the microbial agent, the antigen protein (S1B protein) secreted by Lactococcus lactis adheres to the mucosal surface of the host's cells, forming an antigen protein biofilm on the mucosal surface. The antigen protein binds to the viral binding site on the target cell, blocking the viral receptor protein sites on the mucosal surface. This acts as an ecological barrier. When live viruses in the environment invade the host, the virus binding sites on the target cell are completely blocked by the antigen protein biofilm, preventing the virus from binding to the virus binding sites on the target cells. As a result, the binding of the virus to the receptor on the cell surface is effectively interrupted, providing a preventive effect against PED.

Compared with vaccines, the microbial agent developed in the present disclosure is safer, more effective, and faster. The safety of the microbial agent is demonstrated by secretion of viral functional proteins from the active ingredient, with no viral genes present, thus avoiding viral mutations. Its effectiveness is shown by the secretion of protective antigens from the active ingredient, which acts on the mucosal surface of the host and cover the mucosa surface where the target cells of PEDV are located. When the virus invades, the binding sites on the mucosal surface cells are blocked, thereby interrupting the infection path of virus. The rapidity is evident as the secreted proteins expressed by Lactococcus lactis directly seizes the receptors where the virus binds to the target cells, thereby blocking the virus infection without requiring an immune response process.

### BRIEF DESCRIPTION OF THE DRAWINGS

To illustrate the embodiments of the present disclosure or the technical solutions in the prior art more clearly, the accompanying drawings required in the examples will be briefly described below.

FIG. 1 shows a plasmid map of the recombinant plasmid PNZ8149-Pnis-PpepN-Usp45-S1B in Example 3;

FIG. 2 shows an agarose gel electrophoresis image of the amplified target band of S1B in Example 3;

FIG. 3 shows an agarose gel electrophoresis image of the double digested plasmid vector PNZ8149-Pnis-PpepN-Usp45 and S1B in Example 3;

FIGS. 4A-4C show positive bacterial solution of the recombinant Lactococcus lactis strain with the PNZ8149-Pnis-PpepN-Usp45-S1B in Example 3 and agarose gel electrophoresis images showing the PCR verification on positive colonies;

FIG. 5 shows an Western blot image of the S1B protein in the recombinant Lactococcus lactis strain in Example 4;

FIGS. 6A-6B show the changes in OD value and pH value of the bacterial solution of the recombinant Lactococcus lactis strain in Example 5; and

FIG. 7 shows the diarrhea rate and mortality rate of pigs treated with the bacterial solution of the recombinant Lactococcus lactis strain in Example 9.

### DETAILED DESCRIPTION OF THE EMBODIMENTS

The present disclosure provides a recombinant vector for expressing an immunogenic protein of a PEDV, including a plasmid vector and a nucleotide sequence encoding an S1B protein.

In the present disclosure, the plasmid vector preferably includes PNZ8149-Pnis-PpepN-Usp45, the PNZ8149-Pnis-PpepN-Usp45 is preferably based on a PNZ8149 vector, the PNZ8149 vector preferably includes an original promoter P NisinA, the original promoter P NisinA preferably includes a nucleotide sequence encoding a PpepN promoter and a nucleotide sequence encoding a Usp45 secretion signal peptide that are ligated in sequence downstream; the nucleotide sequence encoding the PpepN promoter is preferably set forth in SEQ ID NO: 2; the nucleotide sequence encoding the Usp45 secretion signal peptide is preferably set forth in SEQ ID NO: 3. The PpepN promoter can initiate the expression of genes located downstream thereof, and can be used for the efficient expression of endogenous or exogenous proteins in prokaryotes; the original promoter P NisinA and the PpepN promoter form a dual promoter combination, which can still express the target protein without adding a Nisin inducer. There is no particular limitation on the method for constructing the recombinant vector, and a conventional method for constructing a recombinant vector may be used.

In the present disclosure, the nucleotide sequence encoding the S1B protein is preferably set forth in SEQ ID NO: 1, and the nucleotide sequence encoding the S1B protein is preferably located between restriction sites Nco I and Sac I of the PNZ8149-Pnis-PpepN-Usp45. The PED S1B protein is a viral spike protein involved in adsorption and entry into target cells.

The present disclosure further provides a recombinant strain expressing the immunogenic protein of PEDV, including the recombinant vector.

In the present disclosure, a host strain for the recombinant strain preferably includes Lactococcus lactis, and the Lactococcus lactis preferably includes Lactococcus lactis NZ3900. There is no particular limitation on the method for constructing the recombinant strain, and conventional methods for constructing recombinant strain may be used.

The present disclosure further provides a microbial agent for preventing PEDV infection, including the recombinant strain as an active ingredient. In the present disclosure, the microbial agent is preferably an oral preparation; the recombinant strain in the microbial agent has a viable count of preferably 2.0-3.0×10⁹ CFU/mL to 2.0-3.0×10¹⁰ CFU/mL, more preferably 2.0-3.0×10¹⁰ CFU/mL. The active ingredient of the microbial agent can secrete and express viral immunogenic proteins, with no viral gene present, avoiding viral mutations, and showing desirable safety. The protective antigens secreted by the active ingredient of the microbial agent act on the mucosal surface of the host, covering the mucosal surface where the target cells are located; when the virus invades, the binding sites on the mucosal surface cells are blocked, so is the viral infection path, resulting in desirable effectiveness. The secretory protein expressed by Lactococcus lactis directly seizes the receptors that the virus would use to bind to the target cells to block viral infection, without the need for an immune response process, showing rapidness.

The present disclosure further provides use of the recombinant vector, the recombinant strain, or the microbial agent in preparation of a drug for preventing PEDV infection. In the present disclosure, the drug preferably includes a drug for blocking binding of the PEDV to a cell receptor.

In order to further illustrate the present disclosure, the recombinant vector for expressing an immunogenic protein of PEDV, the recombinant strain including the same, and use thereof provided by the present disclosure are described in detail below with reference to the accompanying drawings and examples, but the accompanying drawings and the examples should not be construed as limiting the protection scope of the present disclosure.

Unless otherwise specified, the reagents and test materials used in the examples of the present disclosure are all commercially available products. For example, restriction endonucleases Nco I and Sac I can be purchased from NEB; Taq enzyme, dNTP, DNA Marker DL2000, DL15000, Agarose Gel DNA Purification Kit, and Mini BEST Plasmid Purification Kit can be purchased from Takara Biotechnology (Dalian) Co., Ltd.; Eliker reagent can be purchased from Beijing Solarbio Science & Technology Co., Ltd.; GM17 culture medium can be purchased from Qingdao Hi-Tech Industrial Park Hope Bio-Technology Co., Ltd.; cloning vector pNZ8149, NZ9000 Lactococcus, and Lactococcus lactis NZ3900 can be provided by Bio Sci Bio, and lysozyme (CL6951) can be purchased from Beijing Coolaber.

### Example 1

### Acquisition of PED S1B Gene Sequence

The PED S1B protein is a viral spike protein involved in adsorption and entry into target cells. According to the amino acid sequence of the S1B region of PEDV (SJ2008 strain) in NCBI Genbank (Genbank Accession Number: AF353511), the nucleic acid sequence corresponding to amino acids 510 to 640 in the S1B region was codon-optimized for synthesis. The modified sequence was synthesized in full by Sangon Biotech (Shanghai) Co., Ltd., and the sequence information is set forth in SEQ ID NO: 1:

5′-AATATTACTGTTAGTGCTGCTTTTGGTGGTCATAGTGGTGCTAATTTAATTGC
TAGTGATACTACTATTAATGGTTTTAGTAGTTTTTGTGTTGATACTCGGCAATTTACGAT
TACTTTATTTTATAATGTTACTAATAGTTATGGTTATGTTAGTAAAAGTCAAGATAGTAAT
TGTCCATTTACGTTACAAAGTGTTAATGATTATTTATCATTTAGTAAATTTTGTGTTAGT
ACTAGTTTATTAGCCGGTGCTTGTACTATTGATTTATTTGGTTATCCAGAATTTGGTTCA
GGTGTTAAATTTACTAGTTTATATTTTCAATTTACTAAAGGTGAATTAATTACTGGTACT
CCAAAACCATTACAAGGTGTTACTGATGTTAGTTTTATGCATCATCATCATCACCAT-3′.

### Example 2

### Construction of PNZ8149-Pnis-PpepN-Usp45 Plasmid Vector

Primers were designed to amplify the PpepN promoter fragment using NZ9000 Lactococcus as a template, and the nucleotide sequence of the Usp45 secretion signal peptide was amplified using the pVE5523 vector as a template. The nucleotide sequence of the amplified PpepN promoter fragment is set forth in SEQ ID NO: 2, and the forward and reverse primers for amplifying the PpepN promoter fragment were PpepN-F and PpepN-R, respectively; the nucleotide sequence of the amplified Usp45 secretion signal peptide is set forth in SEQ ID NO: 3; the forward and reverse primers for amplifying the Usp45 secretion signal peptide were Usp45-F and Usp45-R, respectively. Specific sequences are as follows:

PpepN promoter sequence (SEQ ID NO: 2):
5′-CTGTCAGTAGACAGTTTTTTTAATAAGTTAAAGAAAAGATGTAATTTTTCTT
TGTACTCGAAATTTTCTATTCAATTTGATATAATTATATTAATACTGAATATTTAGGAGAA
GGC-3′;
PpepN-F (SEQ ID NO: 4):
5′-ATAAGGAGGCACTCACCATGGCTGTCAGTAGACAGTTTTTT-3′;
PpepN-R (SEQ ID NO: 5):
5′-GAGATAATCTTTTTTTTCATGCCTTCTCCTAAATATTCAGTATTAATATAAT-3′;
The Sequence encoding the Usp45 secretion signal peptide (SEQ ID NO: 3):
5′-ATGAAAAAAAAGATTATCTCAGCTATTTTAATGTCTACAGTGATACTTTCTGC
TGCAGCCCCGTTGTCAGGTGTTTACGCTGACACAAACTCAGATATTGCTAAACAAGAT
GCG-3′;
Usp45-F (SEQ ID NO: 6):
5′-ATATTCGGAGGAATTTTGAAATGAAAAAAAAGATTATCTC-3′;
Usp45-R (SEQ ID NO: 7):
5′-TCGTCGTCATCCTTGTAATCCGCATCTTGTTTAGCAATAT-3′.

The PpepN promoter fragment and the Usp45 secretion signal peptide fragment were fused into a PpepN-Usp45 long fragment by overlapping polymerase chain reaction (PCR). The sequence of the PpepN-Usp45 long fragment is set forth in SEQ ID NO: 8, where positions 1 bp to 116 bp corresponds to the PpepN promoter sequence, and 117 bp to 230 bp corresponds to the sequence encoding the Usp45 secretion signal peptide.

PpepN-Usp4 (SEQ ID NO: 8):
CTGTCAGTAGACAGTTTTTTTAATAAGTTAAAGAAAAGATGTAATTTT
TCTTTGTACTCGAAATTTTCTATTCAATTTGATATAATTATATTAATA
CTGAATATTTAGGAGAAGGCATGAAAAAAAAGATTATCTCAGCTATTT
TAATGTCTACAGTGATACTTTCTGCTGCAGCCCCGTTGTCAGGTGTTT
ACGCTGACACAAACTCAGATATTGCTAAACAAGATGCG.

The system for overlapping PCR included: 25 μL of 2×pfu-PCR Master Mix, 2.0 μL each of 10 μm/L forward and reverse primers, 1.0 μL of PpepN promoter fragment, 1.0 μL of Usp45 secretion signal peptide fragment, and supplementing with deionized water to 50 μL.

PCR conditions included: initial denaturation at 98° C. for 5 min; denaturation at 98° C. for 30 s, annealing at 56° C. for 30 s, and extension at 72° C. for 30 s, for a total of 35 cycles; extension at 72° C. for 5 min, and storage at 4° C. after the PCR.

The cloning vector pNZ8149 and the PpepN-Usp45 long fragment are double-digested with Nco I and Xbal I, respectively; the double-digested pNZ8149 and the PpepN-Usp45 long fragment were ligated by T4 ligase to obtain a recombinant plasmid PNZ8149-Pnis-PpepN-Usp45.

### Example 3

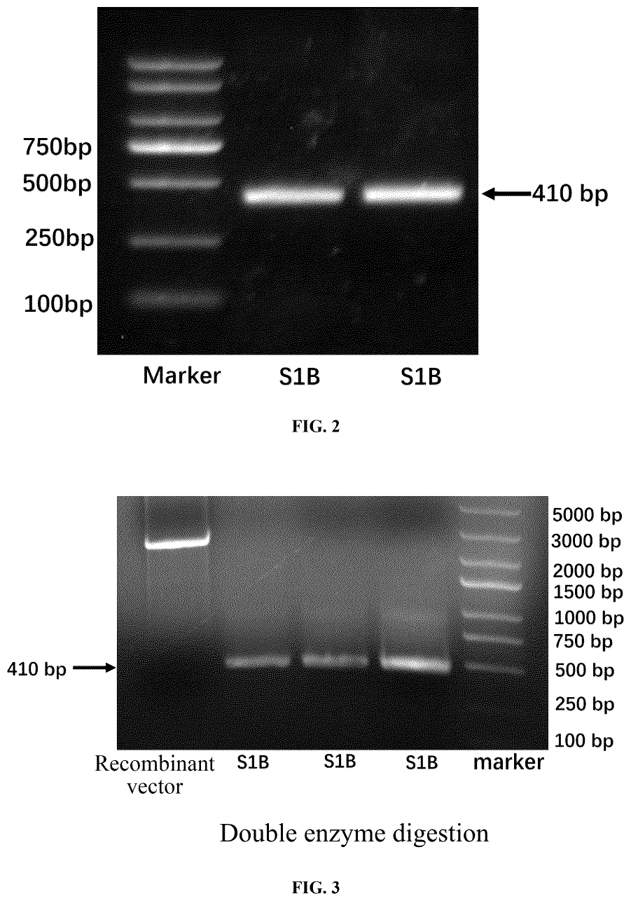

The S1B gene fragment (SEQ ID NO: 1) in Example 1 was used as a template to amplify the S1B gene fragment, with 2 replicates, and the detection results are shown in FIG. 2. As shown in FIG. 2, after the S1B gene fragment was amplified, the S1B gene fragment was successfully obtained by gel recovery.

The primer sequences for amplifying the S1B gene fragment are as follows:

S1B-F (SEQ ID NO: 9):
5′-GATTACAAGGATGACGACGATAAGAATATTACTGTTAGTGCTGCT
TTTG-3′;
S1B-R (SEQ ID NO: 10):
5′-TTAATGGTGATGATGATGATGC-3′.

The recombinant vector plasmid PNZ8149-Pnis-PpepN-Usp45 and S1B gene sequence obtained in Example 2 were double-digested with Nco I and Sac I, respectively. The gel electrophoresis results after the double-digestion of the vector and S1B gene sequence are shown in FIG. 3, where the three S1B lanes corresponds to repeated experiments. As shown in FIG. 3, the PNZ8149-Pnis-PpepN-Usp45 and S1B gene were successfully double-digested, allowing for the next step ligation. The recovered digestion product was ligated with T4 DNA Ligase at 16° C. overnight, and the ligation product was taken out and equilibrated at 4° C. for 4 h, resulting in the recombinant vector PNZ8149-Pnis-PpepN-Usp45-S1B.

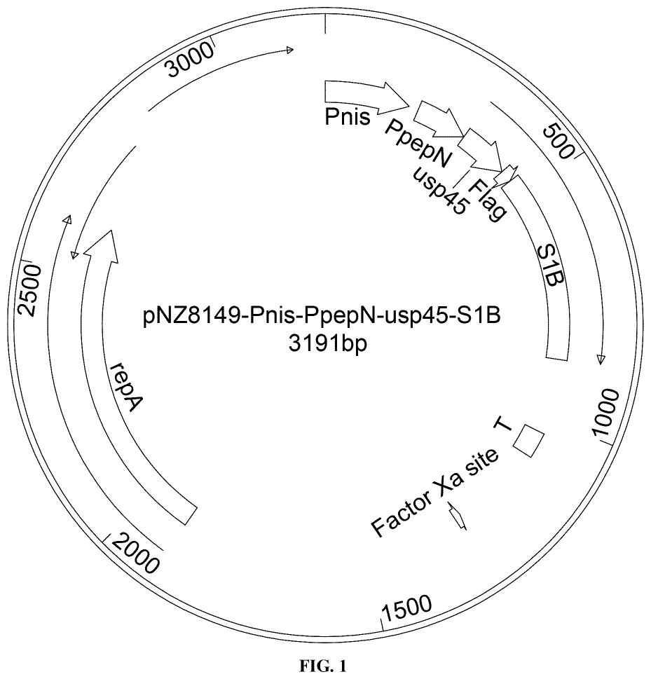

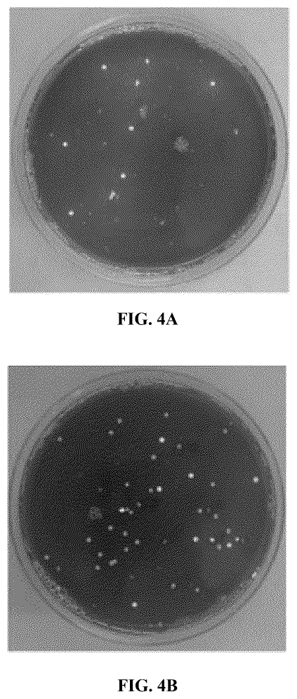

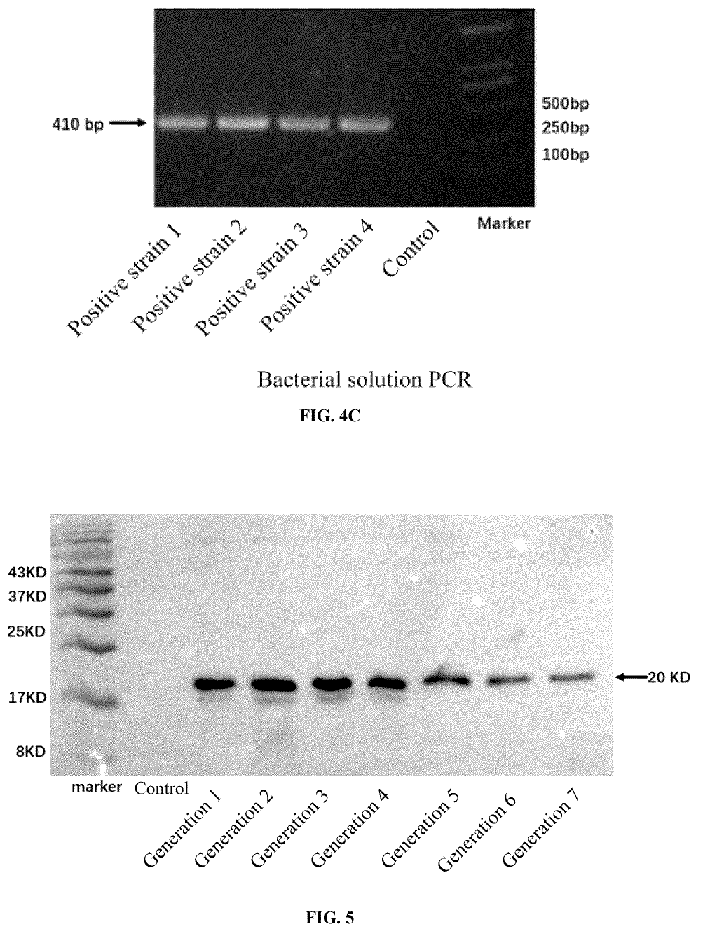

Next, 20 μL of the recombinant vector PNZ8149-Pnis-PpepN-Usp45-S1B was added into competent NZ3900, and incubated on ice for 5 min. The mixture was pipetted into an electroporation curvette, and electroporated for 5 ms. After added into 800 μL of GM17 medium and mixed well, the solution was pipetted into a 1.5 mL newly pre-cooled centrifuge tube, allowed to stand on ice for 5 min, and cultured in a constant-temperature incubator at 30° C. for 4 h. Then 75 μL of Lactococcus lactis NZ3900 cells were spread onto a Eliker solid medium plate. The bacterial cells were statically cultured in a constant-temperature incubator at 30° C. for 16 h. The culture results are shown in FIG. 4A and FIG. 4B, where yellow colonies in FIG. 4A and FIG. 4B were positive colonies. Four positive yellow colonies were selected and cultured in GM17 medium, followed by plasmid extraction and PCR identification. The PCR results are shown in FIG. 4C, where the target fragment appeared at 410 bp. In addition, the plasmids of the 4 positive strains were extracted and sent to Sangon Biotech (Shanghai) Co., Ltd. for sequencing. The sequencing results of the 4 strains with the S1B gene sequence (SEQ ID NO: 1) were subjected to sequence alignment using DNAstar. The sequencing results showed that the sequeces of the 4 positive strains were consistent with the S1B gene sequence (SEQ ID NO: 1). The S1B gene was successfully inserted into the PNZ8149-Pnis-PpepN-Usp45 recombinant vector, and successfully constructed the recombinant plasmid was named PNZ8149-Pnis-PpepN-Usp45-S1B. The recombinant plasmid map is shown in FIG. 1. The resulting positive strain was identified as the recombinant Lactococcus lactis strain.

### Example 4

Construction of Recombinant Lactococcus lactis Strain Expressing S1B Protein and Verification of Stable Protein Expression

The recombinant Lactococcus lactis strain obtained in Example 3 was inoculated into GM17 liquid medium at a ratio of 5% by volume, and the fermentation broth was harvested after the culture at 30° C. for 16 h and then stored at 4° C. The bacterial cells were subcultured every 12 h at a 5% inoculation ratio by volume, and the bacterial suspension was collected until the 7th generation. Preparation of the test sample: 1 mL of the bacterial solution was centrifuged at 4° C. for 3 min, a resulting supernatant was discarded, and the bacterial cells were resuspended and washed with 200 μL of phosphate-buffered saline (PBS). After repetition of the above process, 100 μL of 10 mg/mL lysozyme was added into the bacterial pellet. An obtained mixture was placed in a constant-temperature incubator at 37° C. for 30 min, and the lysozyme was inactivated by boiling in boiling water for 5 min to obtain the test sample. The control group consisted of 100 μL of 10 mg/mL lysozyme, which was incubated in a constant-temperature incubator at 37° C. for 30 min, followed by lyzozyme inactivation in boiling water bath for 5 min to obtain the control sample. The test sample and control sample were mixed with 4×SDS Buffer at a volume ratio of 1:3, boiled for 10 min, and identified by Western Blot.
(1) The test sample was added into the prepared gel for Polyacrylamide Gel Electrophoresis (PAGE), and the proteins were separated by electrophoresis. After Sodium Dodecyl Sulfate Polyacrylamide Gel Electrophoresis (SDS-PAGE), proteins of different molecular weights were separated.
(2) Transfer: bands separated by electrophoresis were transferred from the gel to a Polyvinylidene Fluoride (PVDF) membrane using the electrophoretic transfer method to form a transfer structure of “negative electrode-sponge-three layer filter paper-gel-membrane-three layer filter paper-sponge-positive electrode”. The transfer was conducted at a voltage of 100 V for 70 min. After the electric field was applied, the protein was removed from the polyacrylamide gel and adsorbed on the membrane surface.
(3) Blocking and incubation: After the transfer was completed, the PVDF membrane was transferred to an incubation box containing blocking solution, and the membrane was blocked by shaking on a destaining shaker at room temperature for 1 h. The bands were incubated with a Flag antibody (primary antibody) at 4° C. overnight, and then incubated with a secondary antibody for 2 h. After the secondary antibody was recovered, the bands were washed 3 times with Tris Buffered Saline with Tween 20 (TBST).
(4) Development and image analysis: The PVDF membrane was placed flat on a plastic wrap, and 200 μL developing solution was evenly added using a pipette. The development time was adjusted according to the color depth of the bands.

The Western Blot results are shown in FIG. 5. According to FIG. 5, a band appeared at the target size of 20 KD, while no band appeared in the control group, indicating that the recombinant Lactococcus lactis strain successfully expressed the S1B protein, and the protein could be stably expressed to the 7th generation. The results showed that the recombinant Lactococcus lactis strain that stably expressed the S1B protein was successfully constructed.

### Example 5

1. Viable Count and Growth Curve of Recombinant Lactococcus lactis Strain

The recombinant Lactococcus lactis strain obtained in Example 3 was inoculated into GM17 liquid medium at a inoculation ratio of 5% by volume, and the viable count, optical density (OD) value, and pH value were detected after the culture at 30° C. for 16 h.

### (1) Bacterial Solution Sample

After the liquid sample was thoroughly shaken, 100 μL of the sample was drawn into a 2 mL sterile centrifuge tube containing 900 μL of sterile water using a sterile pipette, followed by shaking thoroughly to obtain a 1:10 sample solution.

### (2) Sample Dilution

On a sterile clean bench, 100 μL of the 1:10 sample homogenate was aseptically aspirated with a 200 μL micropipette and slowly injected along the tube wall into a sterile centrifuge tube containing 900 μL of physiological saline (be careful not to let the tip of the pipette touch the diluent), and the test tube was shaken to mix evenly to obtain a 1:100 sample homogenate. Using another 200 μL micropipette tip, the sample homogenate was diluted in 10-fold increments according to the above operations, and a new micropipette tip was used for each incremental dilution.

(3) Counting of Recombinant Lactococcus lactis Strain by Plate Colony Counting

According to the estimation of the viable cells of the sample to be tested, three consecutive dilutions of 1:10⁵, 1:10⁶, and 1:10⁷ were selected. For each dilution, 100 μL of the sample dilution was pipetted into the pre-prepared GM17 agar plate. Two plates were made for each dilution, and glass beads were added such that the plates were rotated to mix the sample evenly. The culture was incubated anaerobically at 30° C. for 16 h, and the number of all colonies on the plate was counted.

### (4) Colony Characteristics

The colonies are white, round, moist on the surface, opaque, and had neat edges.

### (5) Colony Counting

Viable count of Lactococcus lactis (CFU/mL)=average number of Lactococcus lactis colonies×dilution factor. Plates with colony count at 30 CFU to 300 CFU and no spreading colony growth were selected to count the total number of colonies. The number of colonies for each dilution was the average of two plates. The result was expressed as the viable count per milliliter of sample in CFU/mL, with three significant figures.

### (6) Detection of OD and pH Values

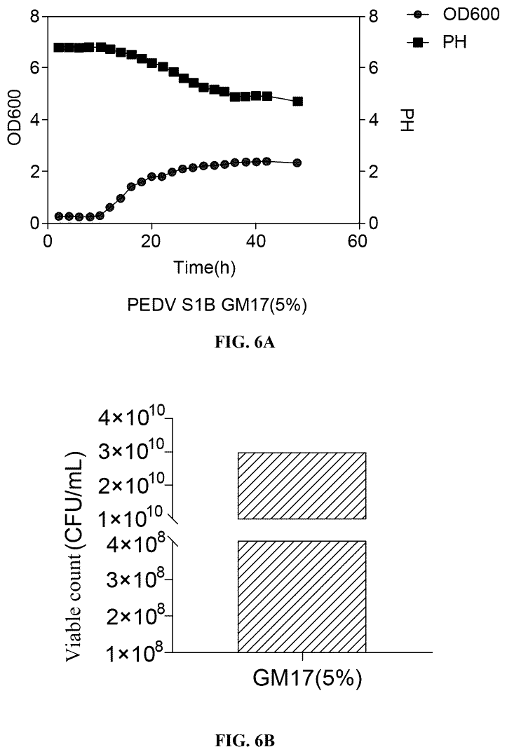

During the culture of recombinant Lactococcus lactis strain, the bacterial solution was taken every 2 h to detect the changes in the OD and pH values of the bacterial solution using a UV spectrophotometer and a pH meter, and detection results are shown in FIG. 6A. When the bacterial concentration and pH value remained basically unchanged, the viable count in the bacterial solution was detected, and detection results are shown in FIG. 6B.

According to FIG. 6A, the recombinant Lactococcus lactis strain was in a logarithmic growth phase during the initial 2 h to 10 h of culture, the bacterial count increased significantly, and the pH value of the bacterial solution also decreased rapidly. The strain entered a stable phase at 14 h to 24 h, and the bacterial concentration and pH value remained basically unchanged. According to FIG. 6B, the viable count of the recombinant Lactococcus lactis strain could reach 3×10¹⁰ CFU/mL after entry into the stable phase. Therefore, the culture time of the recombinant Lactococcus lactis strain was preferably 14 h to 24 h.

2. Preparation of Stock Solution of Recombinant Lactococcus lactis strain

The recombinant Lactococcus lactis strain obtained in Example 3 was inoculated into GM17 liquid medium at a inoculation ratio of 5% by volume, and cultured at 30° C. for 16 h, resulting in a stock solution of the recombinant Lactococcus lactis strain, namely the culture stock solution, which was then stored at 4° C. in refrigerator. The viable count of the culture stock solution was 3×10¹⁰ CFU/mL.

3. Preparation of Concentrate of Recombinant Lactococcus lactis Strain

One milliliter of the stock solution was centrifuged at 6,000 rpm for 5 min in a high-speed centrifuge at 4° C., the supernatant was removed, and 200 μL of sterile physiological saline was added to allow re-suspension. resulting in a concentrated solution of the recombinant Lactococcus lactis strain, namely the culture concentrate, which was stored in a refrigerator at 4° C. for future use. The viable count of the culture concentrate was 1.5×10¹¹ CFU/mL.

### 4. Preparation of Medium Stock Solution

Firstly, 4.2 g of the finished M17 medium powder and 0.5 g of glucose were added into 100 mL of pure water, and then sterilized in an autoclave at 121° C. for 20 min to obtain the medium stock solution, which was cooled and stored in a refrigerator at 4° C. for later use.

### 5. Preparation of Medium Concentrate

After 5 times weighing of the original dosage of the finished M17 medium, 21 g of the finished M17 medium powder and 2.5 g of glucose were added into 100 mL of pure water, sterilized in an autoclave at 115° C. for 20 min to obtain the medium concentrate, which was cooled and stored in a refrigerator at 4° C. for later use.

### Example 6

Acute Toxicity of Recombinant Lactococcus lactis Strain to Mice

Eighty healthy 6-week-old Specific Pathogen Free (SPF) Kunming mice, half male and half female, were randomly divided into 4 groups, with 10 female mice and 10 male mice in each group. The groups were recorded as a medium stock solution control group, a medium concentrate control group, a recombinant Lactococcus lactis stock solution group (culture stock solution group), and a recombinant Lactococcus lactis concentrate group (culture concentrate group).

The medium stock solution, medium concentrate, recombinant Lactococcus lactis stock solution, and recombinant Lactococcus lactis concentrate prepared in Example 5 were placed at room temperature for 10 min, and were orally gavaged into mice at a dose of 20 mL/kg BW for 3 consecutive days. The mice were observed for 21 d, and counted for toxic reactions during the observation period. The results are shown in Table 1. As shown in Table 1, no toxic reaction or death was observed in the mice after they took the recombinant Lactococcus lactis strain, indicating that the recombinant Lactococcus lactis strain was non-pathogenic and highly safe.

TABLE 1
Acute toxicity effect of recombinant Lactococcus lactis strain to mice

| | Toxic reactions in female animals | | | | | |
|---|---|---|---|---|---|---|
| | Number | | | Toxic reactions in male animals | | |
| | of | Number of | Number | Number of | Number of | Number |
| | animals | reaction | of death | animals | reaction | of death |
| Group | (mice) | (mice) | (mice) | (mice) | (mice) | (mice) |
| Medium stock | 10 | 0 | 0 | 10 | 0 | 0 |
| solution control | | | | | | |
| group | | | | | | |
| Culture stock | 10 | 0 | 0 | 10 | 0 | 0 |
| solution group | | | | | | |
| Medium concentrate | 10 | 0 | 0 | 10 | 0 | 0 |
| control group | | | | | | |
| Culture concentrate | 10 | 0 | 0 | 10 | 0 | 0 |
| group | | | | | | |

### Example 8

Production of recombinant Lactococcus lactis preparation: the recombinant Lactococcus lactis strain obtained in Example 3 was inoculated into 10 L of GM17 liquid medium at a inoculation ratio of 5% by volume, and cultured at 30° C. for 16 h, resulting in a recombinant Lactococcus lactis preparation, which was then stored at 4° C. in refrigerator. The viable count of the recombinant Lactococcus lactis preparation was 3×10¹⁰ CFU/mL.

A total of 367 piglets and 50 farrowing sows (7-15 d before farrowing) in a pig farm in Kaifeng City, Henan Province were tested positive for PEDV infection. Newborn piglets developed diarrhea one after another, and the emergency immunization with vaccines and drug treatment yielded unsatisfactory results. After the onset of disease, 50 farrowing sows (7-15 d before farrowing) were gavaged with 10 mL of the recombinant Lactococcus lactis preparation every day; while 367 prematurely weaned piglets were gavaged with 5 mL of the recombinant Lactococcus lactis preparation every day. On the next day of administration of the recombinant Lactococcus lactis preparation, the symptoms of piglets over 10 d old were significantly alleviated; and after administration at the above dosage for 5 consecutive days, the diarrhea symptoms of all piglets were effectively controlled, with a total of 10 casualties, showing a mortality rate of 2.8%. The piglets born from the farrowing sows treated with the recombinant Lactococcus lactis preparation no longer had diarrhea. This indicated that the recombinant Lactococcus lactis preparation was effective.

### Example 9

Preparation of recombinant Lactococcus lactis preparation: the recombinant Lactococcus lactis strain obtained Example 3 was inoculated into 10 L of GM17 liquid medium at a inoculation ratio of 5% by volume, and cultured at 30° C. for 16 h, resulting in a recombinant Lactococcus lactis preparation, which was then stored at 4° C. in refrigerator. The viable count of the recombinant Lactococcus lactis preparation was 3×10¹⁰ CFU/mL.

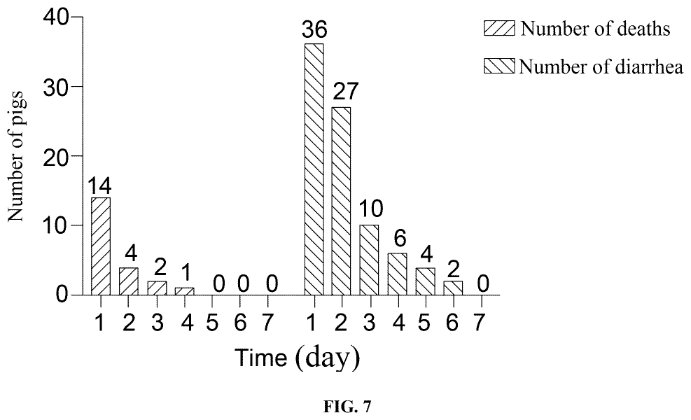

In a pig farm in Hunan, 50 piglets aged 32-37 d had diarrhea symptoms, which were confirmed to be caused by PEDV infection. The piglets had been ill for 5 days prior to the administration of the recombinant Lactococcus lactis preparation. Each piglet was gavaged with 10 mL of the recombinant Lactococcus lactis preparation every day, and the number of pig deaths and pig diarrhea was counted every day. The results are shown in FIG. 7. As shown in FIG. 7, by the third day of using the recombinant Lactococcus lactis preparation, the number of pig deaths per day in the pig herd dropped from 14 to 2, while the number of pigs with diarrhea per day dropped from 36 to 10; by the 7th day of using the recombinant Lactococcus lactis preparation, both the mortality and diarrhea counts in the pig herd dropped to 0. This indicates that the recombinant Lactococcus lactis preparation is effective in significantly reducing diarrhea rates and mortality rate.

Although the above examples have described the present disclosure in detail, it is only a part of, not all of the embodiments of the present disclosure. Other embodiments may also be obtained by persons based on the examples without creative efforts, and all of these examples shall fall within the protection scope of the present disclosure.

What is claimed is:

1. A recombinant vector for expressing an immunogenic protein of a porcine epidemic diarrhea virus (PEDV), comprising a plasmid vector and a nucleotide sequence encoding an S1B protein.

2. The recombinant vector according to claim 1, wherein the plasmid vector comprises PNZ8149-Pnis-PpepN-Usp45; and
the PNZ8149-Pnis-PpepN-Usp45 is based on a PNZ8149 vector and comprises a nucleotide sequence encoding a PpepN promoter and a nucleotide sequence encoding a Usp45 secretion signal peptide; wherein the nucleotide sequence encoding the PpepN promoter and the nucleotide sequence encoding the Usp45 secretion signal peptide are ligated in sequence and located downstream of an original promoter of the PNZ8149 vector; the nucleotide sequence encoding the PpepN promoter is set forth in SEQ ID NO: 2; and the nucleotide sequence encoding the Usp45 secretion signal peptide is set forth in SEQ ID NO: 3.

3. The recombinant vector according to claim 2, wherein the nucleotide sequence encoding the S1B protein is located between restriction sites Nco I and Sac I of the PNZ8149-Pnis-PpepN-Usp45.

4. A recombinant strain expressing an immunogenic protein of PEDV, comprising the recombinant vector according to claim 1.

5. The recombinant strain according to claim 4, wherein the plasmid vector comprises PNZ8149-Pnis-PpepN-Usp45; and
the PNZ8149-Pnis-PpepN-Usp45 is based on a PNZ8149 vector and comprises a nucleotide sequence encoding a PpepN promoter and a nucleotide sequence encoding a Usp45 secretion signal peptide; wherein the nucleotide sequence encoding the PpepN promoter and the nucleotide sequence encoding the Usp45 secretion signal peptide are ligated in sequence and located downstream of an original promoter of the PNZ8149 vector; the nucleotide sequence encoding the PpepN promoter is set forth in SEQ ID NO: 2; and the nucleotide sequence encoding the Usp45 secretion signal peptide is set forth in SEQ ID NO: 3.

6. The recombinant strain according to claim 5, wherein the nucleotide sequence encoding the S1B protein is located between restriction sites Nco I and Sac I of the PNZ8149-Pnis-PpepN-Usp45.

7. The recombinant strain according to claim 4, wherein a host strain for the recombinant strain comprises Lactococcus lactis.

8. The recombinant strain according to claim 7, wherein the Lactococcus lactis comprises Lactococcus lactis NZ3900.

9. A microbial agent for preventing PEDV infection, comprising the recombinant strain according to claim 4 as an active ingredient.

10. The microbial agent according to claim 9, wherein a host strain for the recombinant strain comprises Lactococcus lactis.

11. The microbial agent according to claim 10, wherein the Lactococcus lactis comprises Lactococcus lactis NZ3900.

12. The microbial agent according to claim 9, wherein the recombinant strain in the microbial agent has an effective viable count of 2.0-3.0×10⁹ colony forming unit (CFU)/mL to 2.0-3.0×10¹⁰ CFU/mL.

13. A method for preventing PEDV infection, comprising administering to a subject in need thereof a therapeutic effective amount of a drug comprising the recombinant vector according to claim 1.

14. The method according to claim 13, wherein the drug comprises a drug for blocking binding of the PEDV to a cell receptor.
